(19) United States
(12) Patent Application Publication
(10) Pub. No.: US 2021/0259758 A1
(43) Pub. Date: Aug. 26, 2021

(54) SURGICAL CARDIAC TISSUE ABLATION CLAMP

(71) Applicant: Tamim Antaki, Indialantic, FL (US)

(72) Inventor: Tamim Antaki, Indialantic, FL (US)

(21) Appl. No.: 17/184,731

(22) Filed: Feb. 25, 2021

Related U.S. Application Data
(60) Provisional application No. 62/981,076, filed on Feb. 25, 2020.

(51) Int. Cl. A61B 18/08

(52) U.S. Cl. CPC A61B 18/085; A61B 2018/00982

(57) ABSTRACT

A system for cardiac ablation including a first Jaw, second jaw, shaft, and joint. The first jaw having a longitudinal axis. The second jaw having a longitudinal axis parallel to the longitudinal axis of the first jaw. The shaft having a longitudinal axis parallel to the longitudinal axis of the first jaw and the longitudinal axis of the second jaw. The joint secured to the first jaw, the second jaw, and shaft. The joint is operable to position the first jaw and second jaw in an open position or a closed position. The longitudinal axes of the first jaw, second jaw, and shaft are parallel in both the open position and closed position.

### RELATED APPLICATIONS

This application claims priority under 35 U.S.C. §119(e) of U.S. Provisional Patent Application Ser. No. 62/981,076 (Attorney Docket No. 4693.00001) filed on Feb. 25, 2020 and titled SURGICAL CARDIAC TISSUE ABLATION CLAMP The content of this application is incorporated herein by reference.

### FIELD OF THE INVENTION

The present invention relates to systems and methods for cardiac ablation. More specifically, the present invention relates to a system and method for cardiac tissue ablation for management of cardiac arrhythmias with particular benefit for atrial arrhythmias including atrial fibrillation and atrial flutter

### BACKGROUND OF THE INVENTION

Atrial fibrillation is a common cardiac arrhythmia characterized by an irregular, typically fast, heart rhythm. Potential negative effects on an atrial fibrillation patient's quality of life include risk of thromboembolic strokes due to blood clot formation in the heart, progressive deterioration of cardiac pump function, and shortened lifespan. Current management of atrial fibrillation includes administration of anticoagulation agents to reduce the risk of blood clot formation in the heart, thereby reducing risk of thromboembolic stroke, administration of antiarrhythmic medications to manage heart rhythm, direct-current electrical cardioversion, and specifically focused and measured coagulative or deep hypothermic injury to certain locations inside atrial cardiac muscle in order to trap Irregular electrical cardiac activity, suppress it, and eliminate the source or re-channel the activity to prevent electrical cardiac stimulus reentry. The latter is termed ablation therapy and typically utilizes multiple energy delivery sources in order to heat up cardiac tissue. Typical energy sources include radiofrequency electrical current, microwave energy, laser energy, and localized deep hypothermia, referred to here as ablation forces.

A variety of tools, including suction assisted catheters and catheter like tools inserted either endovascularly or extracardiac, have been developed to deliver the above-mentioned ablation forces. A sustained and well proven design of reproducible energy delivery has been In the form of a variety of clamps, which compress the intended cardiac atrial tissue between two jaws of the clamp and deliver bipolar electrical current at a certain radiofrequency effecting localized heating of the tissue and inducing coagulation of vital proteins and cessation of electrophysiologic properties of that part of cardiac tissue. The reproducibility of energy delivery with minimalization of area of intended injury reinforce the validity of the clamp model as a cardiac ablation tool.

Cardiac tissue ablation has been established as a mainline therapy and treatment for atrial fibrillation and other atrial arrhythmias. Bipolar principles of ablation have been established as being superior to unipolar principles of ablation regardless of the type of energy delivered to perform the ablation. Bipolar methods provide more consistency and higher reproducibility of lesions, which may account for the better safety profile associated with bipolar methods. Similarly, bipolar methods reduce collateral damage to unintended adjacent tissues and structures.

Multiple designs of bipolar energy delivery devices have been in clinical use and have been granted U.S. patents. Known bipolar energy delivery devices include an L-shaped or right-angled configuration between the clamp and the shaft of the device. Additionally, available bipolar energy devices provide limited flexibility and rotational ability between the shaft and the clamp. Prior art clamps have been in the form of fixed right-angled configurations with limited rotation between the handle and the shaft and between the shaft and the clamp jaws with no rotation in orthogonal or angular planes. These prior art designs limit the utility of the clamps and their use during minimal invasive surgical approaches. Known bipolar energy delivery devices have been intended for open surgical approaches, in which the chest cavity is opened with a large incision going through the middle of the sternum or in between the ribs. These known designs are not compatible with the trends towards minimally Invasive, port based, and robotic assisted thoracoscopic surgical interventions.

Introduction of such clamps through small port-like 5 to 15 mm incisions in the trunk wall and subsequent maneuvering and manipulation of those devices Inside the chest cavity is fraught with limitations and could potentially be hazardous. Therefore, a need exists for a new design of cardiac ablation clamp in order to facilitate utility during minimal invasive, port based, and robotic approaches.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a system for cardiac ablation including a first and second jaw, a shaft, and a joint. The first jaw may have a longitudinal axis. The second jaw may have a longitudinal axis parallel to the longitudinal axis of the first jaw. The shaft may have a longitudinal axis parallel to the longitudinal axis of the first jaw and the longitudinal axis of the second jaw. The joint may be secured to the first jaw, the second jaw, and shaft. The joint may be operable to position the first jaw and second jaw in an open position or a closed position. The longitudinal axes of the first jaw, second jaw, and shaft may be parallel in both the open position and closed position.

The shaft may include a hollow, outer housing and an inner housing slidably earned within the outer housing. The shaft may further include a spring having a first end secured to the outer housing and a second end secured to the inner housing.

The system may include a first handle secured to a first end of the outer housing and a second handle secured to a first end of the inner housing.

The joint may secure to a second end of the inner housing.

The joint may include a plurality of elongate members. The plurality of elongate members may include a first elongate member having a first end secured to a first end of the first jaw and a second end secured to the second end of the inner housing, a second elongate member having a first end secured to a first end of the second jaw and a second end secured to the second end of the inner housing, a third elongate member having a first end secured to a length of the first jaw and a second end secured to the second end of the inner housing, and a fourth elongate member having a first end secured to a length of the second jaw and a second end secured to the second end of the inner housing. The third elongate member may be parallel to the first elongate member and the fourth elongate member may be parallel to the second elongate member.

The system may include an actuator adapted to maintain the first jaw and the second jaw in a closed position.

The system may further include a first energy conductor pad located on a face of the first jaw and a second energy conductor pad located on a face of the second jaw.

The system may include a first locking mechanism located on a second end of the first jaw and a second locking mechanism located on a second end of the second jaw.

The system may further include an actuator adapted to activate the first locking mechanism.

### BRIEF DESCRIPTION OF THE DRAWINGS

Some embodiments of the present invention are illustrated as an example and are not limited by the figures of the accompanying drawings, in which like references may indicate similar elements.

FIG. 1 is a front elevation view of a cardiac ablation clamp in an open configuration according to one embodiment of the present Invention.

FIG. 2 is a side elevation view of the cardiac ablation clamp of FIG. 1.

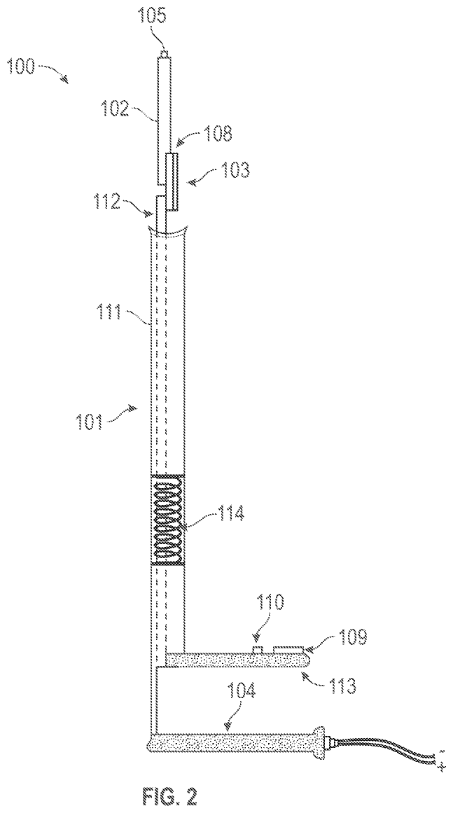

FIG. 3 is a front elevation view of a cardiac ablation clamp in a closed configuration according to one embodiment of the present invention.

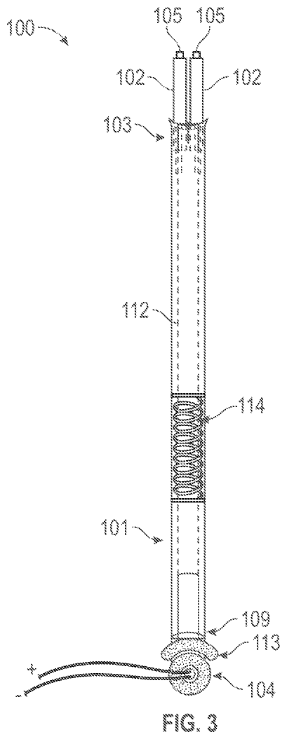

FIG. 4 is a side elevation view of the cardiac ablation clamp of FIG. 3.

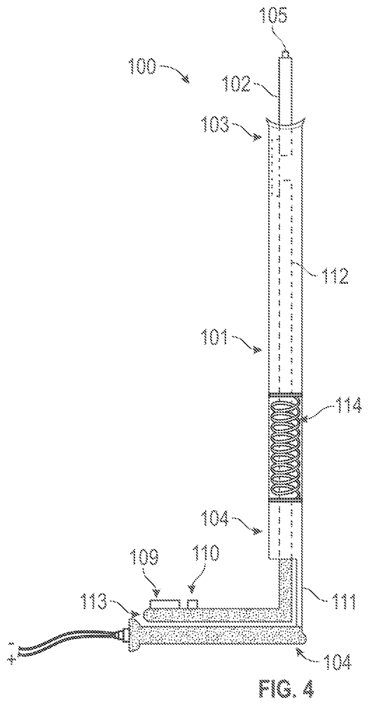

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

The cardiac ablation clamp 100 is specifically intended for cardiac tissue ablation The cardiac ablation clamp 100 has utility in atrial tissue ablation for the treatment of cardiac arrhythmias and may be used to treat atrial fibrillation, atrial flutter, and similar atrial arrhythmias. The cardiac ablation clamp 100 may be used while the heart is beating and without cardiopulmonary bypass support. However, the cardiac ablation damp 100 may also be compatible with use in surgeries involving cardiopulmonary bypass with full cardiac and respiratory support or in procedures involving cardiac arrest as well.

The straight design of the clamp 100 allows for easy insertion and removal of the cardiac ablation damp 100 into and out of the thoracic cavity. The straight design of the clamp 100 also provides easy navigation around vital anatomical cardiovascular, neurologic, and pulmonary structures in the tight space of the chest with minimal disturbance of tissues This straight-line configuration also allows consistent delivery of ablation energy to intended areas. Most importantly, the straight-line design of the cardiac ablation clamp 100 allows bipolar delivery of energy providing full thickness lesions while all known contact based and suction based technologies fall short of delivering consistent full thickness lesions

An embodiment of the invention, as shown and described by the various figures and accompanying text, provides a cardiac ablation clamp 100 having a linearly oriented, straight design The damp 100 may include a shaft 101 secured to jaws 102 in a straight-line configuration. The jaws 102 may be oriented in line with the shaft 101 to form a 180-degree angle between a longitudinal axis of the shaft 101 and a longitudinal axis of the jaws 102 when the jaws 102 are in a neutral, non-open position. A joint 103 may secure the jaws 102 to the shaft 101 and allow the jaws 102 to be moved along a single axis from the neutral position to an open position to form an angle of deflection with respect to the shaft 101, which may range between 150 degrees and 210 degrees.

The shaft 101 may include two nested components. An outer, hollow housing 111 may be secured to a fixed handle 104 at a first end An inner housing 112 may be carried within the hollow housing 111 and secured to a slidable handle 113 at a first end. The inner housing 112 may be carried by the outer housing 111 in a configuration that allows the inner housing 112 to slide along a length of the outer housing 111 extending along the longitudinal axis of the outer housing 111. The outer housing 111 and inner housing 112 may be oriented with parallel, or coincident, longitudinal axes The outer housing 111 and inner housing 112 may have coincident centers.

A spring 114 may be positioned and configured to secure the inner housing 112 to the outer housing 111 to maintain the slidable handle 113 spaced apart from the fixed handle 104 when the spring 114 is in its neutral, relaxed stated. Squeezing the fixed handle 104 and the slidable handle 113 toward one another may elongate the spring 114 and position the inner housing 112 closer to the fixed handle 104 or a first end of the outer housing 111, thereby causing a second end of the inner housing 112 to retract into or toward the opening of the outer housing 111 at the second end of the outer housing 111, which is distal the fixed handle 104. Removing an external force from the fixed handle 104 and slidable handle 113 may result in the force of the spring 114 moving the handles 113, 104 away from one another, which causes the second end of the inner housing 112 to extend past the opening of the outer housing 111 at the second end of the outer housing 111.

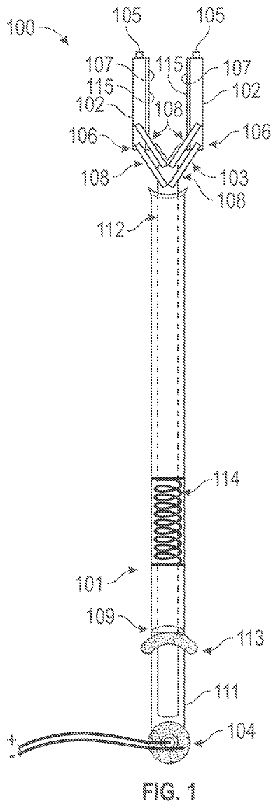

In one embodiment, a multi-faceted joint mechanism 103 may secure the jaws 102 to the second end of the inner housing 112. The joint mechanism 103 may allow for a deflection of a longitudinal axis of each jaw 102 of about 30 degrees. The longitudinal axes of the jaws 102 may each travel within the same plane, with the faces of each jaw 102 remaining in two separate, perpendicular planes, at least as depicted in FIG. 1.

The design of the cardiac ablation clamp 100 provides a straight configuration, which allows easy introduction inside the human body through a small 8 to 15 mm port site as well as in-line delivery around cardiac structures with minimal disturbance. The cardiac ablation clamp 100 may have a deflection angle of up to 30 degrees. This deflection angle may allow a first end 106 of each jaw 102 to travel between 0 degrees and 30 degrees with respect, to a plane extending along the longitudinal axis of the shaft 101. A face 107 of a first jaw 102 may lay along the longitudinal axis on a first Side and a face 107 of a second jaw 102 may lay along the longitudinal axis on a second Side, which the face 107 of each jaw 102 opposing the other, when the clamp 100 is in a closed position. The face 107 of each jaw 102 may travel away from the longitudinal axis of the shaft 101 when the clamp 100 is moved to an open position. The face 107 of each jaw 102 may remain horizontal to the face 107 of the opposing jaw 102 while moving from the closed to the open position In such an embodiment, the actuator 109 may be activated to maintain the jaws 102 in the retracted position or may be released to allow the jaws 102 to transition to the opened position.

The joint 103 may include a plurality of elongate members 108. Each elongate member 108 may have a first end secured to the shaft 101 and a second end secured to a jaw 102. Each elongate member 108 may be actuated to rotate with respect to the shaft 101. In one embodiment: each elongate member 108 may be configured to rotate up to thirty degrees with respect to the shaft 101. The angle of rotation of the elongate member 108 with respect to a longitudinal axis of the shaft 101 may be referred to as the angle of deflection. An actuator 109 may be carried by the shaft 101 or a portion of the handle 104. The actuator 109 may be adjusted to position the plurality of elongate members 108, resulting in continuous positionability of the jaws 102 between the open and closed positions. The actuator 109 may actuate deflection of the elongate members 108 using a dial incorporated as part of the handle 104 of the device or otherwise carried by the clamp 100. In embodiments in which the actuator 109 is a dial, rotating the actuator 109 in a first direction may bring the faces 107 of the jaws 102 closer together while rotating the actuator in an opposite, second direction may move the faces 107 of the jaws 102 away from one another.

In another embodiment, the jaws 102 may transition between the open and closed positions by using a trigger-type handle configuration. In such an embodiment, the slidable handle 113 may be retracted toward the fixed handle 104, which retracts the jaws 102 into the hollow cavity of the outer housing 111. The outer housing 111 may be sized to have an interior width equal to or slight greater than the width of the two jaws 102. Retracting the jaws 102 into the outer housing 111, may force the jaws into the closed position. Springs may be positioned on the joint 103 to force the jaws into the opened position when the joint 103 is positioned exterior the outer housing 111.

The jaws 102 may include a distal locking mechanism 105 allowing them to latch onto a cooperatively configured locking mechanism connected to the tips of rubber ropes or hollow tubes (rubber elements). The locking mechanism 105 may be reversible to release the rubber elements from the jaws 102 and allow for repositioning of the clamp 100. After the clamp 100 is repositioned to the opposite side of the chest, the rubber elements may be re-latched to the jaws 102. The distal locking mechanism 105 may be activated or deactivated to lock or unlock the locking mechanism 105. A distal locking mechanism 105 actuator 110 may be located on the handle 104, along the shaft 101, or otherwise carried by the clamp 100. The actuator 110 may be activated to lock the locking mechanism 105, securing the locking mechanism 105 to cooperatively configured components positioned proximate the locking mechanism 105 and deactivated to unlock the locking mechanism 105, releasing the cooperatively configured components secured to the locking mechanism 105.

The clamp may be operable 100 by inserting the jaws 102 into a thoracic cavity. The jaws may be positioned on opposing sides of a portion of cardiac, or other, tissue to be ablated Ablation energy may be provided to the tissue in the form of radiofrequency energy delivered through conducting elements running down from an input point at the proximal end of the device and extending down the shaft 101 to each of the jaws 102 of the clamp 100. The ablation energy may be administered to the tissue through one or more energy conductor pads 115 located on a surface of the jaws 102. Other potential sources of energy include microwave energy, laser energy, a mechanism to deliver deep hypothermic cryogenic elements on each jaw 102 of the clamp 100, or the like. The jaws 102 may be positioned in an open configuration to insert the tissue between the faces 107 of the jaws 102 and then moved toward a closed position to securely capture the tissue between the jaws 102, in an engaged position. While in the engaged position, ablation energy may be delivered to at least a portion of the face 107 of each jaw 102 through an energy conductor pad 115 to ablate the tissue in contact with the energy conductor pad 115.

The cardiac ablation clamp 100 may be used by an inventive method. The cardiac ablation clamp 100 may be used in minimal invasive surgical treatment of atrial fibrillation, atrial flutter, other cardiac rhythm abnormalities using port access into the pericardial cavity and chest cavities, including right and left thoracic cavities and the pericardial sac. One inventive method involves the use of an endoscopic camera loaded on a scope, which may be 5-7 millimeters. The scope may be inserted through a camera port, which may be less than a centimeter, located in the right thoracic cavity. Two additional ports, which may be 5-12 millimeters, may be located proximate to the camera port on the right side of the thoracic cavity The first additional port may be located anatomically superior to the camera port. The second additional port may be located anatomically inferior to the camera port. These additional ports may be utilized to introduce robotic and/or thoracoscopic instruments. Another port, which may be 8-15 millimeter wide port and referenced as device port, may be inserted on the lateral side of the right thoracic cavity anatomically posterior to the camera port. This port may be utilized to introduce the inventive cardiac ablation clamp. Using the robotic/thoracoscopic camera inserted in the camera port and robotic/thoracoscopic surgical instruments, which may be inserted in either the first or second additional port, the pericardial reflection between inferior vena cava and the right inferior pulmonary vein may be incised. Such an incision would open the oblique sinus of the pericardial sac. The pericardial reflection between superior vena cava and the upper surface of the left atrium and pulmonary artery and the soft tissues above the dome of the left atrium and the pericardial reflection between left superior pulmonary vein and left pulmonary artery may also be severed by blunt dissection aided by the robotic and thoracoscopic camera to allow visualization of the left atrial appendage. Such dissection may establish what is anatomically described as the transverse sinus Under thoracoscopic vision, two magnetically tipped semi-flexible catheters, referenced as magnetic guides, may be introduced into the oblique and transverse sinuses from superior and inferior ports on the right side of the torso and allowed to couple magnetically on the left side of the left atrium inside the pericardial sac. This connection of the magnetic guides across the sinuses provides continuity around the far side of left atrium. Using the coupled magnetic guides back and forth, these guides may be utilized to deliver two hollow, multi-perforated rubber tubes or solid easy-glide ropes, referred here as rubber elements, which may be 3-6 millimeters wide, around the left atrium. Each one of the rubber elements may be color coded. One end of each of the two rubber elements may be retrieved from the 8-15 millimeter device port. The other end of each rubber element may be kept protruding from each of the superior or inferior ports, respectively. This configuration may establish a pulley mechanism. The two ends of different rubber elements emerging from the device port may latch to the distal ends of referenced inventive ablation clamp by a reversible, locking mechanism By applying tension to the ends of the rubber elements emerging from the superior and inferior ports the pulley effect will advance the inventive ablation clamp 100 through the device port and will induce one jaw of the clamp to advance inside the transverse sinus and the other jaw inside the oblique sinus of the pericardial sac. The left atrial wall will become enclosed between the two clamp jaws 102. In order to decrease heat spread and unintended collateral heating of adjacent tissues and organs, the rubber elements may also function as irrigation catheters. Each rubber element may be in the form of a tube with multiple apertures located through a thickness of the sidewalls to allow an infusion of cold saline solution running down the exteriorized ends thus preventing unintended collateral heating.

Each of the two ends of the rubber elements emerging from the 8-15 millimeter device port may have a reversible locking latch mechanism, which may each secure to a suitable locking mechanism 105 at the tip of the jaws 102 of the cardiac ablation clamp 100. The rubber elements may be secured to the cardiac ablation clamp jaws 102 using the reversible locking latch mechanism. The cardiac ablation clamp jaws 102 may be Introduced into the chest cavity through the 8-15 millimeter port and be guided around the left atrial wall by applying tension to the end of either rubber element protruding from the superior and inferior ports Once the cardiac ablation clamp 100 is positioned, the handle 104 of the clamp 100 may be squeezed to activate the jaws 102 causing them to move toward one another and clamp down on either side of a portion of the left atrium. Energy, which may include, but is not limited to radiofrequency, microwave, deep hypothermic cryogenic forceps, or the like, may be introduced to the damp jaws 102 to perform a bipolar full-thickness ablation. The steps of utilizing the rubber elements to position the jaws 102, clamping a portion of the left atrium, and applying energy to perform an ablation may be repeated as necessary to obtain full thickness and consistent linear lesions of ablation of the targeted cardiac tissue.

In order to decrease collateral heat spread, cold normalized saline solution may be infused down one or both ends of the hollow rubber elements during the ablation or at the completion of ablation sessions to decrease risk of inadvertent adjacent tissue and organ injury.

To achieve complete isolation of atrial fibrillation sources in the left atrium this surgical method may be repealed going to the left side of the thorax. The rubber elements may be disengaged from the ablation clamp 100 after completion of ablation of the right side. The ends of each of the rubber elements may then be clipped to its corresponding other end thus making a form of two complete rings around the left atrium. The ends of rubber elements may be retrieved when the ablation procedure is moved to the left thoracic cavity using identical steps and ports. The same inventive steps may be repeated on the left side in order to generate a complete circle of linearly ablated cardiac tissue running through the left atrial muscle wall from the front of the right pulmonary veins to the front of the left pulmonary veins. This complete ablation line may provide electrical isolation of the posterior surface of the left atrium and all of the pulmonary veins, which constitute the majority of sources of atrial fibrillation irregular cardiac activity.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the Invention will Include all embodiments falling within the scope of the appended claims. Also, in the drawings and the description, there have been disclosed exemplary embodiments of the Invention and, although specific terms may have been employed, they are unless otherwise stated used in a generic and descriptive sense only and not for purposes of limitation, the scope of the invention therefore not being so limited Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. A system for cardiac ablation comprising:
a first jaw having a longitudinal axis;
a second jaw having a longitudinal axis parallel to the longitudinal axis of the first jaw;
a shaft having a longitudinal axis parallel to the longitudinal axis of the first jaw and the longitudinal axis of the second jaw; and
a joint secured to the first jaw, the second jaw, and shaft;
wherein the joint is operable to position the first jaw and second jaw in an open position or a closed position; and
wherein the longitudinal axes of the first jaw, second jaw, and shaft are parallel in both the open position and closed position.

2. The system of claim 1 wherein the shaft further comprises:
a hollow, outer housing; and
an inner housing slidably carried within the outer housing

3. The system of claim 2 wherein the shaft further comprises:
a spring having a first end secured to the outer housing and a second end secured to the inner housing

4. The system of claim 2 further comprising:
a first handle secured to a first end of the outer housing; and
a second handle secured to a first end of the inner housing.

5. The system of claim 2 wherein the joint secures to a second end of the inner housing.

6. The system of claim 5 wherein the joint comprises e plurality of elongate members.

7. The system of claim 6 wherein the plurality of elongate members comprises:
a first elongate member having a first end secured to a first end of the first jaw and a second end secured to the second end of the inner housing; and
a second elongate member having a first end secured to a first end of the second jaw and a second end secured to the second end of the inner housing.

8. The system of claim 7 wherein the plurality of elongate members comprises:
a third elongate member having a first end secured to a length of the first jaw and a second end secured to the second end of the inner housing; and
a fourth elongate member having a first end secured to a length of the second jaw and a second end secured to the second end of the inner housing; and
wherein the third elongate member is parallel to the first elongate member and the fourth elongate member is parallel to the second elongate member.

9 The system of claim 7 further comprising an actuator adapted to maintain the first jaw and the second jaw in a closed position.

10. The system of claim 1 further comprising a first energy conductor pad located on a face of the first jaw.

11. The system of claim 10 further comprising a second energy conductor pad located on a face of the second jaw.

12. The system of claim 1 further comprising a first locking mechanism located on a second end of the first jaw.

13. The system of claim 12 further composing a second locking mechanism located on a second end of the second jaw.

14. The system of claim 12 further comprising an actuator adapted to activate the first locking mechanism.

15. A system for ablation comprising:
a first jaw having a longitudinal axis;
a second jaw having a longitudinal axis parallel to the longitudinal axis of the first jaw;
a shaft having a longitudinal axis parallel to the longitudinal axis of the first jaw and the longitudinal axis of the second jaw, comprising,
a hollow, outer housing, and
an inner housing slidably carried within the outer housing;
a joint secured to the first jaw, the second jaw, and a second end of the inner housing:
a first energy conductor pad located on a face of the first jaw;
wherein the joint is operable to position the first jaw and second jaw in an open position or a closed position; and
wherein the longitudinal axes of the first jaw, second jaw, and shaft are parallel in both the open position and closed position.

16. The system of claim 15 wherein the shaft further comprises:
a spring having a first end secured to the outer housing and a second end secured to the inner housing

17. The system of claim 15 further comprising.
a first handle secured to a first end of the outer housing; and
a second handle secured to a first end of the inner housing.

18. The system of claim 15 wherein the joint comprises:
a first elongate member having 3 first end secured to a first end of the first jaw and a second end secured to the second end of the inner housing;
a second elongate member having a first end secured to a first end of the second jaw and a second end secured to the second end of the inner housing;
a third elongate member having a first end secured to a length of the first jaw and a second end secured to the second end of the inner housing; and
a fourth elongate member having a first end secured to a length of the second jaw and a second end secured to the second end of the inner housing; and
wherein the third elongate member is parallel to the first elongate member and the fourth elongate member is parallel to the second elongate member.

19. The system of claim 15 further comprising:
a first locking mechanism located on a second end of the first jaw; and
an actuator adapted to activate the first locking mechanism.

20. A system for ablation comprising:
a first jaw having a longitudinal axis;
a second jaw having a longitudinal axis parallel to the longitudinal axis of the first jaw;
a shaft having a longitudinal axis parallel to the longitudinal axis of the first jaw and the longitudinal axis of the second jaw, further comprising,
a hollow, outer housing,
an inner housing slidably carried within the outer housing, and
a spring having a first end secured to the outer housing and a second end secured to the inner housing;
a first handle secured to a first end of the outer housing;
a second handle secured to a first end of the Inner housing;
a joint secured to the first jaw, the second jaw, and a second end of the inner housing, comprising:
a first elongate member having a first end secured to a first end of the first jaw and a second end secured to the second end of the inner housing,
a second elongate member having a first end secured to a first end of the second jaw and a second end secured to the second end of the inner housing,
a third elongate member having a first end secured to a length of the first jaw and a second end secured to the second end of the inner housing, and
a fourth elongate member having a first end secured to a length of the second jaw and a second end secured to the second end of the inner housing, and
wherein the third elongate member is parallel to the first elongate member and the fourth elongate member is parallel to the second elongate member;
a first energy conductor pad located on a face of the first jaw;
a second energy conductor pad located on a face of the second jaw;
a first locking mechanism located on a second end of the first jaw;
a second locking mechanism located on a second end of the second jaw; and
an actuator adapted to activate the first locking mechanism;
wherein the joint is operable to position the first jaw and second jaw in an open position or a closed position; and
wherein the longitudinal axes of the first jaw, second jaw, and shaft are parallel In both the open position and closed position.
